(19) United States
(12) Patent Application Publication
(10) Pub. No.: US 2025/0020678 A1
(43) Pub. Date: Jan. 16, 2025

(54) VALIDATION DEVICE, VALIDATION METHOD AND VALIDATION PROGRAM

(71) Applicant: SHIMADZU CORPORATION, Kyoto (JP)

(72) Inventors: Satoru WATANABE, Kyoto (JP); Yuma OKABE, Kyoto (JP); Chihiro YASUI, Kyoto (JP); Yusuke YOKOI, Kyoto (JP)

(73) Assignee: SHIMADZU CORPORATION, Kyoto (JP)

(21) Appl. No.: 18/897,733

(22) Filed: Sep. 26, 2024

Related U.S. Application Data
(63) Continuation of application No. 17/278,554, filed on Mar. 22, 2021.

(51) Int. Cl. G01N 35/00

(52) U.S. Cl. CPC G01N 35/00871; G01N 35/00732

(57) ABSTRACT

An inspection item for an analysis unit is specified. A correspondence relationship between the inspection item and an operator code assigned to each operator of the analysis unit is acquired. Operation commands assigned to operators to be operated for execution of an inspection of the analysis unit are sequentially transmitted to the analysis unit based on the inspection item and the correspondence relationship. A request command requesting transmission of screen information representing a screen to be displayed in the display panel of the analysis unit is transmitted to the analysis unit. The screen information that has been transmitted by the analysis unit in response to the request command is acquired. The analysis unit behaves based on the operation command assigned to the operated operator and a screen responding to the behavior of the analysis unit is displayed in the display panel of the analysis unit.

### CROSS-REFERENCE TO RELATED APPLICATIONS

This application is a continuation of U.S. patent application Ser. No. 17/278,554, filed on Mar. 22, 2021, which is a PCT US National Phase of PCT/JP2018/035957 filed on Sep. 27, 2018, the disclosures of each are hereby incorporated herein by reference in their entireties.

### TECHNICAL FIELD

The present invention relates to a validation device, a validation method and a validation program for validating an analysis device.

### BACKGROUND ART

After an analysis device is introduced, it is necessary to validate whether the analysis device behaves normally (see Patent Document 1, for example). Such validation includes validating each analysis unit of an analysis device satisfying the specification at the time of installation and validating each analysis unit of the analysis device satisfying defined performance. Generally, validation of an analysis device is executed when a technician such as a field engineer (hereinafter referred to as a performer) inspects each analysis unit in regard to an inspection item. Further, the performer creates a report that mentions a result of inspection, etc.
[Patent Document 1] JP 2002-340907 A

### SUMMARY OF INVENTION

### Technical Problem

Since a large number of inspection items are included in the above-mentioned validation, it is cumbersome to execute an inspection manually in regard to each of the inspection items. Therefore, validation is desirably automated. In the validation device of the Patent Document 1, an inspection in regard to part of the inspection items can be automated. However, in a case where validation is automated, whether the same inspection as the inspection to be executed manually has been executed properly cannot be easily confirmed, and reliability of validation is lowered. In this manner, a reduction in time and labor for validation conflicts with maintenance of reliability of validation, and it is not easy to meet both of the requirements.

An object of the present invention is to provide a validation device, a validation method and a validation program that enable maintenance of reliability of validation while reducing time and labor for validating an analysis device.

### Solution to Problem

(1) A validation device according to one aspect of the present invention is to validate an analysis unit included in an analysis device, wherein the analysis unit has a plurality of operators, has a display panel that displays a screen for executing an inspection of the analysis unit based on an operation of any of the plurality of operators and is configured to behave based on an operation command assigned to an operated operator, and the validation device includes an item specifier that specifies an inspection item for the analysis unit, a correspondence relationship acquirer that acquires a correspondence relationship between the inspection item and an operator code assigned to each operator of the analysis unit, an operation command transmitter that sequentially transmits operation commands assigned to operators to be operated for execution of an inspection of the analysis unit to the analysis unit based on the inspection item and the correspondence relationship, a request command transmitter that transmits a request command requesting transmission of screen information representing a screen to be displayed in the display panel to the analysis unit, and a screen information acquirer that acquires the screen information that has been transmitted by the analysis unit in response to the request command.

In this validation device, the inspection item for the analysis unit is specified. The correspondence relationship between the inspection item and the operator code assigned to each operator of the analysis unit is acquired. The operation commands assigned to the operators to be operated for execution of an inspection of the analysis unit are sequentially transmitted to the analysis unit based on the inspection item and the correspondence relationship. The request command requesting transmission of screen information representing a screen to be displayed in the display panel is transmitted to the analysis unit. The screen information transmitted by the analysis unit in response to the request command is acquired. The screen responding to the behavior of the analysis unit is displayed in the display panel of the analysis unit.

With this configuration, it is not necessary for the performer who manages the inspection of the analysis unit to manually perform at least part of work for inspecting the analysis unit. This reduces time and labor for validating the analysis device. Further, the performer can identify that operation commands are sequentially received by the analysis unit and the procedure for inspecting the analysis unit proceeds in response to the received operation commands, by viewing the transition of screens displayed in the display panel of the analysis unit. Therefore, whether the same inspection as the inspection to be executed manually has been executed properly can be easily confirmed. As a result, it is possible to maintain reliability of validation while reducing time and labor for validating the analysis device.
(2) The request command transmitter may transmit a next request command to the analysis unit each time an operation command is transmitted by the operation command transmitter, and the display panel may update a screen displayed based on a received operation command each time an operation command transmitted by the operation command transmitter is received.

In this case, a next operation command is transmitted to the analysis unit while it is confirmed that an operator corresponding to each operation command has been operated. Thus, even in a case where a response from the analysis unit is delayed, a series of operation commands can be transmitted to the analysis unit easily and sequentially while an operator is being operated.
(3) The request command transmitter may discard a transmitted request command and may retransmit a same request command in a case where screen information is not acquired by the screen information acquirer in a certain period of time after the request command is transmitted. With this configuration, even in a case where a response from the analysis unit is delayed, the screen information can be received from the analysis unit.
(4) The analysis device may include a plurality of analysis units, and the validation device may further include an order determiner that determines order of inspections of the plurality of analysis units based on priority levels indicating the order of inspections of the plurality of analysis units, and the operation command transmitter, the request command transmitter and the screen information acquirer may behave such that the inspections of the plurality of analysis units are executed based on the order determined by the order determiner. In this case, the plurality of analysis units can be inspected efficiently based on the priority levels.
(5) The validation device may further include a result determiner that acquires a result of inspection of the analysis unit and a report creator that creates a report mentioning a result of inspection acquired by the result acquirer. In this case, it is not necessary for the performer to manually create a report that mentions a result of inspection. This reduces time and labor for the performer more sufficiently. Further, an error in transferring a result of inspection and so on can be prevented.
(6) A validation method according to another aspect of the present invention is to validate an analysis unit included in an analysis device, wherein the analysis unit has a plurality of operators, has a display panel that displays a screen for executing an inspection of the analysis unit based on an operation of any of the plurality of operators and is configured to behave based on an operation command assigned to an operated operator, and the validation method includes the steps of specifying an inspection item for the analysis unit, acquiring a correspondence relationship between the inspection item and an operator code assigned to each operator of the analysis unit, sequentially transmitting operation commands assigned to operators to be operated for execution of an inspection of the analysis unit to the analysis unit based on the inspection item and the correspondence relationship, transmitting a request command requesting transmission of screen information representing a screen to be displayed in the display panel to the analysis unit, and acquiring the screen information that has been transmitted by the analysis unit in response to the request command.

With this validation method, it is not necessary for the performer who manages the inspection of the analysis unit to execute at least part of manual work for inspecting the analysis unit. This reduces time and labor for validating the analysis device. Further, the performer can identify that operation commands are sequentially received by the analysis unit and the procedure for inspecting the analysis unit proceeds in response to the received operation commands, by viewing the transition of screens displayed in the display panel of the analysis unit. Therefore, whether the same inspection as the inspection to be executed manually has been executed properly can be easily confirmed. As a result, it is possible to maintain reliability of validation while reducing time and labor for validating the analysis device.
(7) A validation program according to yet another aspect of the present invention is to validate an analysis unit included in an analysis device, wherein the analysis unit has a plurality of operators, has a display panel that displays a screen for executing an inspection of the analysis unit based on an operation of any of the plurality of operators and is configured to behave based on an operation command assigned to an operated operator, and the validation program causes a processing device to execute the processes of specifying an inspection item for the analysis unit, acquiring a correspondence relationship between the inspection item and an operator code assigned to each operator of the analysis unit, sequentially transmitting operation commands assigned to operators to be operated for execution of an inspection of the analysis unit to the analysis unit based on the inspection item and the correspondence relationship, transmitting a request command requesting transmission of screen information representing a screen to be displayed in the display panel to the analysis unit, and acquiring the screen information that has been transmitted by the analysis unit in response to the request command.

With this validation program, it is not necessary for the performer who manages the inspection of the analysis unit to execute at least part of manual work for inspecting the analysis unit. This reduces time and labor for validating the analysis device. Further, the performer can identify that operation commands are sequentially received by the analysis unit and the procedure for inspecting the analysis unit proceeds in response to the received operation commands, by viewing the transition of screens displayed in the display panel of the analysis unit. Therefore, whether the same inspection as the inspection to be executed manually has been executed properly can be easily confirmed. As a result, it is possible to maintain reliability of validation while reducing time and labor for validating the analysis device.

### ADVANTAGEOUS EFFECTS OF INVENTION

It is possible to maintain reliability of validation while reducing time and labor for validating the analysis device.

### BRIEF DESCRIPTION OF THE DRAWINGS

FIG. 1 is a diagram showing the configuration of an analysis system including a validation device according to one embodiment of the present invention.

FIG. 2 is a diagram showing the appearance of an analysis unit.

FIG. 3 is a time chart showing the basis behavior of the validation device;

FIG. 4 is a diagram showing one example of an initial screen displayed in a display in a validation process.

FIG. 5 is a diagram showing one example of an execution screen for an inspection in the validation process.

FIG. 6 is a diagram showing another example of the execution screen for an inspection in the validation process.

FIG. 7 is a diagram showing one example of a report representing a result of inspection.

FIG. 8 is a diagram showing the configuration of the validation device.

FIG. 9 is a flowchart showing the algorithm of the validation process to be executed by a validation program.

FIG. 10 is a flowchart showing the algorithm of the validation process to be executed by the validation program.

### DESCRIPTION OF EMBODIMENTS

### (1) Configuration of Analysis System

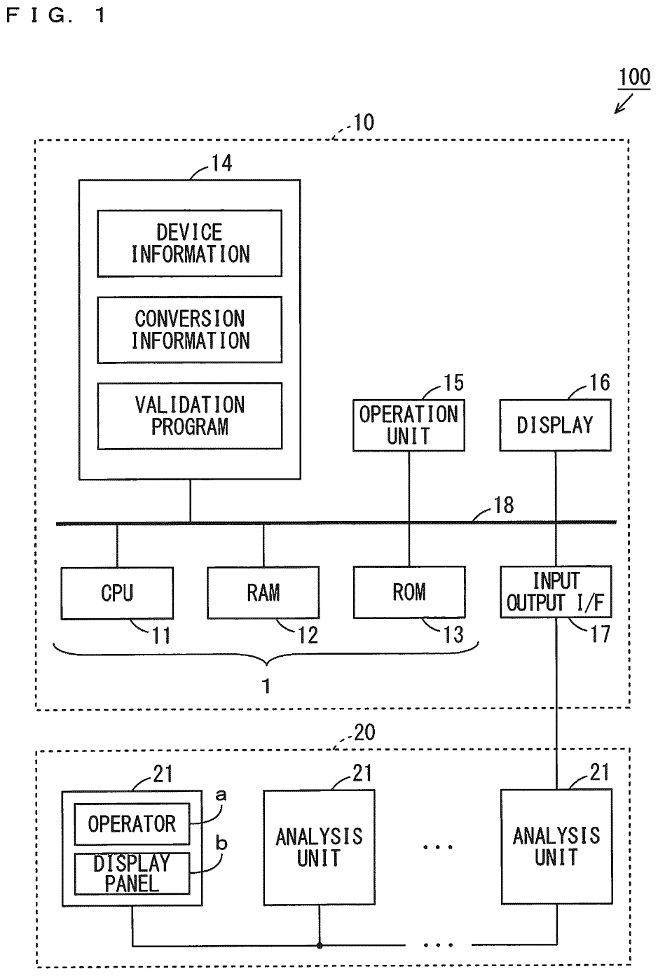

A validation device, a validation method and a validation program according to embodiments of the present invention will be described below in detail with reference to the drawings. FIG. 1 is a diagram showing the configuration of an analysis system including the validation device according to one embodiment of the present invention. As shown in FIG. 1, the analysis system 100 includes an analysis control device 10 and an analysis device 20. The analysis system 100 may include a plurality of analysis devices 20.

The analysis control device 10 is constituted by a CPU (Central Processing Unit) 11, a RAM (Random Access Memory) 12, a ROM (Read Only Memory) 13, a storage 14, an operation unit 15, a display 16 and an input output I/F (interface) 17. The CPU 11, the RAM 12, the ROM 13, the storage 14, the operation unit 15, the display 16 and the input output I/F 17 are connected to a bus 18. The CPU 11, the RAM 12 and the ROM 13 constitute the validation device 1.

The RAM 12 is used as a work area for the CPU 11. A system program is stored in the ROM 13. The storage 14 includes a storage medium such as a hard disc or a semiconductor memory. The validation program is stored in the storage 14. The validation program may be stored in a storage medium different from the storage 14. The CPU 11 executes the validation program stored in the storage 14 or the like on the RAM 12, whereby a validation process is executed. Details of the validation process will be described below. Further, device information and conversion information, described below, are stored in the storage 14.

The operation unit 15 is an input device such as a keyboard, a mouse or a touch panel. The display 16 is a display device such as a liquid crystal display device. A performer, described below, can provide various instructions to the validation device 1 using the operation unit 15. The display 16 displays an execution screen for the validation process to be executed by the validation device 1, and so on. The input output I/F 17 is connected to the analysis device 20.

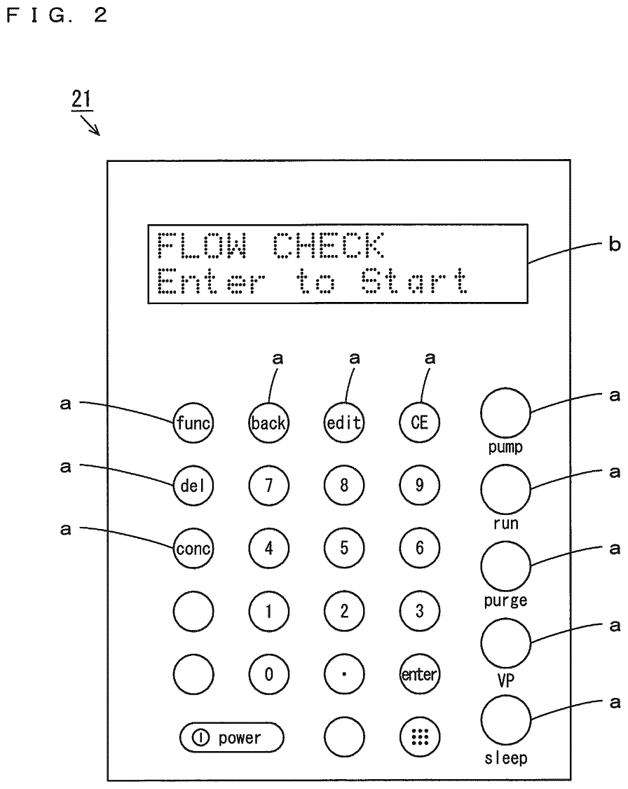

The analysis device 20 is a liquid chromatograph or a gas chromatograph, for example, and includes analysis units 21 such as a pump, an autosampler, a column oven, a detector and a system controller. FIG. 2 is a diagram showing the appearance of an analysis unit 21.

As shown in FIG. 2, each analysis unit 21 has a plurality of operators ‘a’ that receive operations performed by a user and a display panel b that displays a character string. The operators ‘a’ include predetermined function keys and ten keys. The plurality of operators ‘a’ and the display panel b may be implemented by a touch panel. After the analysis device 20 is introduced, it is necessary to validate whether the analysis device 20 behaves normally. This integrity is validated by execution of an inspection of each analysis unit 21 in regard to a predetermined inspection item.

As the inspection items for a pump, initialization, a firmware version, a liquid sending stability, a pressure limiter, accuracy of flow rate and a leak sensor and so on are included. As the inspection items for an autosampler, initialization, a firmware version, a basic behavior, a leak sensor, accuracy of set temperature of a sample cooler, accuracy of injection rate and so on are included. As the inspection items for a column oven, a firmware version, a temperature adjustment function, a gas sensor, a leak sensor and so on are included. As the inspection items for a detector, initialization, a firmware version, lamp energy, a lamp lighting time, accuracy of wavelength, a leak sensor and so on are included.

Normally, a technician such as a field engineer (hereinafter referred to as a performer) manually executes an inspection of each analysis unit 21. Specifically, the performer performs a predetermined operation on an operator ‘a’ of an analysis unit 21 to be inspected. In this case, a screen for executing an inspection in regard to a desired inspection item is displayed in the display panel b. After operating an analysis unit 21 as necessary, the performer operates one or more operators ‘a’ for executing an inspection in regard to the inspection item.

In the example of FIG. 2, a screen for executing an inspection in regard to the accuracy of flow rate of a pump is displayed in the display panel b. Here, the performer sets a volumetric flask in an analysis unit 21 and causes the analysis unit 21 to behave with the flow rate of 1 mL/min, for example, by operating an operator ‘a.’ Thereafter, the performer measures a period of time it takes for 5 mL, for example, of liquid to be collected in the volumetric flask and inputs the measured time to the analysis unit 21 by operating an operator ‘a.’ The flow rate is calculated by the analysis unit 21 based on the input time. In a case where the calculated flow rate is in an allowable range from a predetermined reference value, it is determined that the analysis unit 21 has “passed” an inspection in regard to the inspection item.

The above-mentioned operation is repeated in regard to other inspection items, whereby the inspection of the analysis unit 21 ends. Thereafter, the performer creates a report that mentions a result of inspection, the name of the performer, inspection execution date and time, etc. in regard to a predetermined analysis unit 21.

The validation device 1 sequentially transmits an operation command for operating one or more operators ‘a’ to be operated for execution of an inspection of each analysis unit 21 and a request command for requesting transmission of screen information representing a screen to be displayed in the display panel b. In this case, the same screens as the screens displayed at the time of a manual inspection are sequentially displayed in the display panel b. Thus, the analysis unit 21 can be inspected while whether the same inspection as the manual inspection has been executed appropriately is presented to the performer or the like. Behavior of the validation device 1 will be described below.

### (2) Behavior of Validation Device

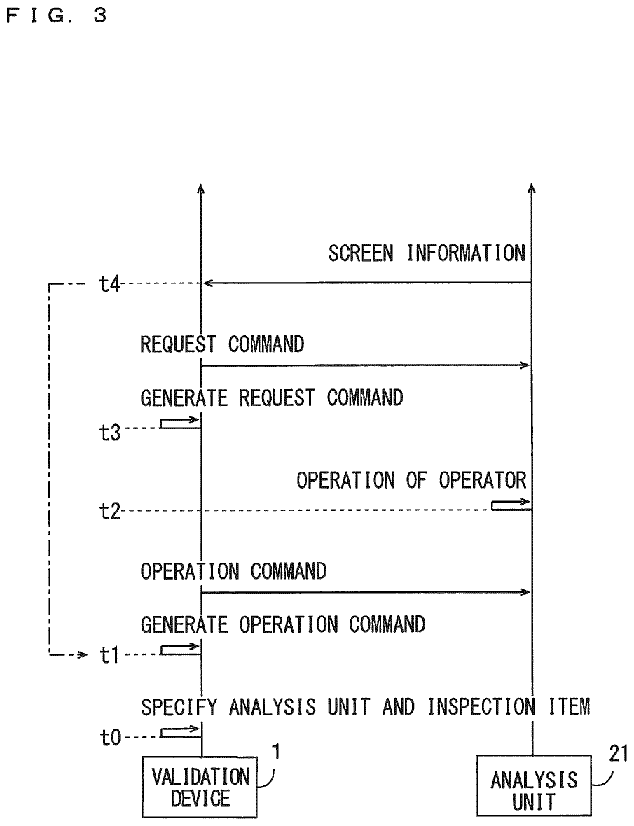

FIG. 3 is a time chart showing the basic behavior of the validation device 1. As shown in FIG. 3, at a point t0 in time, the validation device 1 specifies an analysis unit 21 to be inspected and an inspection item based on device information stored in the storage 14 of FIG. 1. The device information is a PDF (Portable Document Format) file in the present example and represents the configuration of the analysis device 20 connected to the analysis control device 10, an inspection item for each analysis unit 21 included in the analysis device 20 and so on. At the point t0 in time, a predetermined initial screen is displayed in the display panel b of an analysis unit 21.

At a point t1 in time, the validation device 1 generates an operation command for operating an operator ‘a’ of the analysis unit 21 based on an inspection item for the analysis unit 21 and conversion information stored in the storage 14. The conversion information is the information that represents the correspondence relationship between an inspection item and an operator code assigned to each operator ‘a’ of the analysis unit 21 and is unique to each type of an analysis unit 21. Further, the validation device 1 transmits the generated operation command to the analysis unit 21.

At a point t2 in time, the analysis unit 21 operates the operator ‘a’ in response to the operation command received from the validation device 1. Thus, the screen displayed in the display panel b of the analysis unit 21 is updated. At a point t3 in time, the validation device 1 generates a request command and transmits the generated request command to the analysis unit 21. At a point t4 in time, in response to the request command received from the validation device 1, the analysis unit 21 transmits the screen information representing the screen being displayed in the display panel b to the validation device 1.

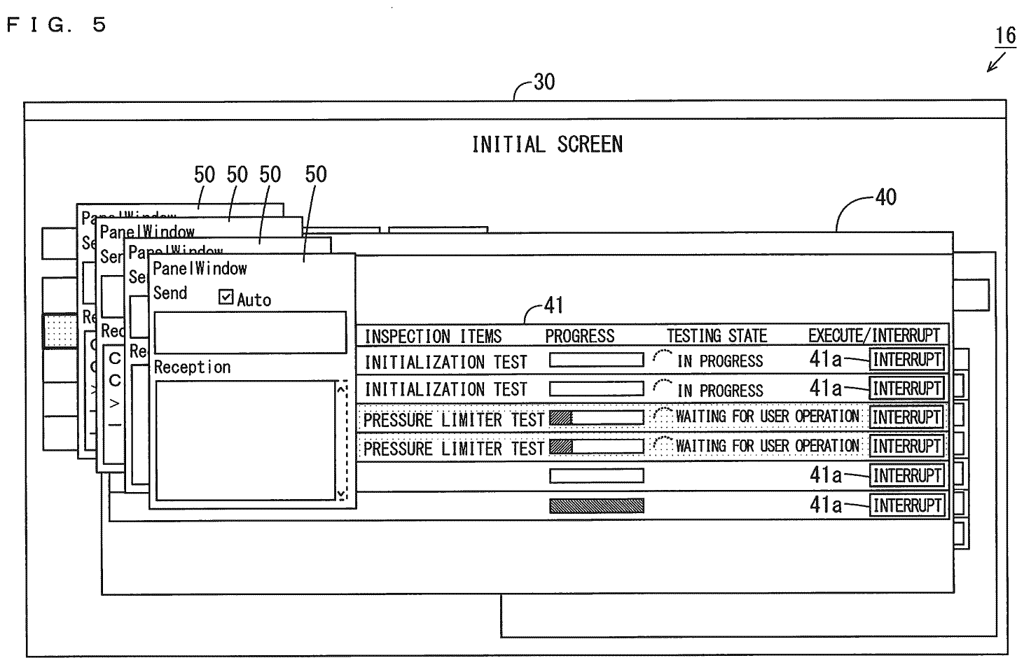

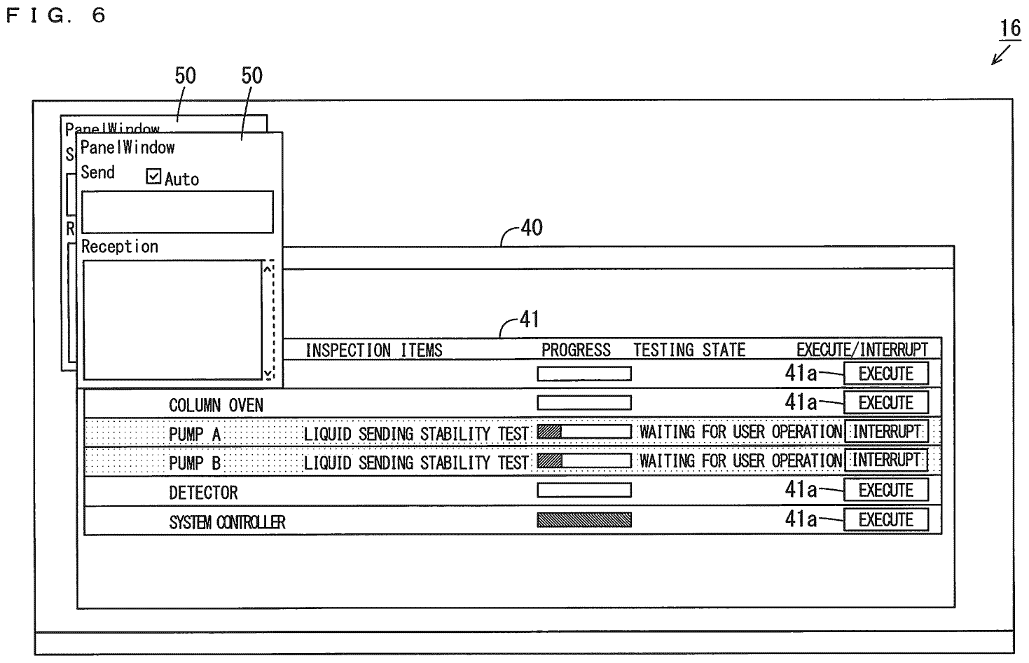

The validation device 1 confirms that the operator ‘a’ is operated at the point t2 in time based on the screen information received from the analysis unit 21 at the point t4 in time. Here, in a case where a simulation screen 50 of FIG. 5 or FIG. 6, described below, is displayed in the display 16, the validation device 1 displays a screen similar to the screen displayed in the display panel b of the analysis unit 21 in the simulation screen 50.

Further, the validation device 1 determines whether the inspection of the analysis unit 21 in regard to all of the inspection items has ended based on the screen information. In a case where the inspection of the analysis unit 21 in regard to all of the inspection items has not ended, the validation device 1 returns to the process executed at the point t1 in time. The process executed at the points t1 to t4 in time is repeated until the inspection of an analysis unit 21 in regard to a predetermined inspection item ends. Further, the analysis unit 21 may be manually operated by the performer before the point t1 in time.

In the present example, in a case where not receiving the screen information from the analysis unit 21 in a certain period of time (ten seconds, for example) after transmitting the request command, the validation device 1 discards the request command and retransmits the same request command. Further, in a case where receiving screen information not responding to the request command, the validation device 1 ignores the screen information.

Specifically, it is considered that the validation device 1 transmits a first request command to the analysis unit 21 at the point t3 in time. Here, suppose that the analysis unit 21 transmits the screen information responding to the first request command to the validation device 1 at the point t4 in time which is twelve seconds after the point t3 in time, that is, a period of time longer than 10 seconds has elapsed from the point t3 in time.

In this case, the validation device 1 discards the first request command and retransmits a second request command that is the same as the first request command to the analysis unit 21 ten seconds after the point t3 in time. Two seconds after that, the validation device 1 receives the screen information responding to the first request command from the analysis unit 21. Thus, the validation device 1 proceeds to the process executed at the point t4 in time. Thereafter, the validation device 1 may receive the screen information responding to the second request command from the analysis unit 21. Even in this case, the validation device 1 ignores the screen information as the screen information does not correspond to the request command.

With this configuration, even in a case where a response from the analysis unit 21 is delayed, the validation device 1 can receive the screen information from the analysis unit 21. Further, a plurality of screen information pieces are prevented from being received in response to a same request command.

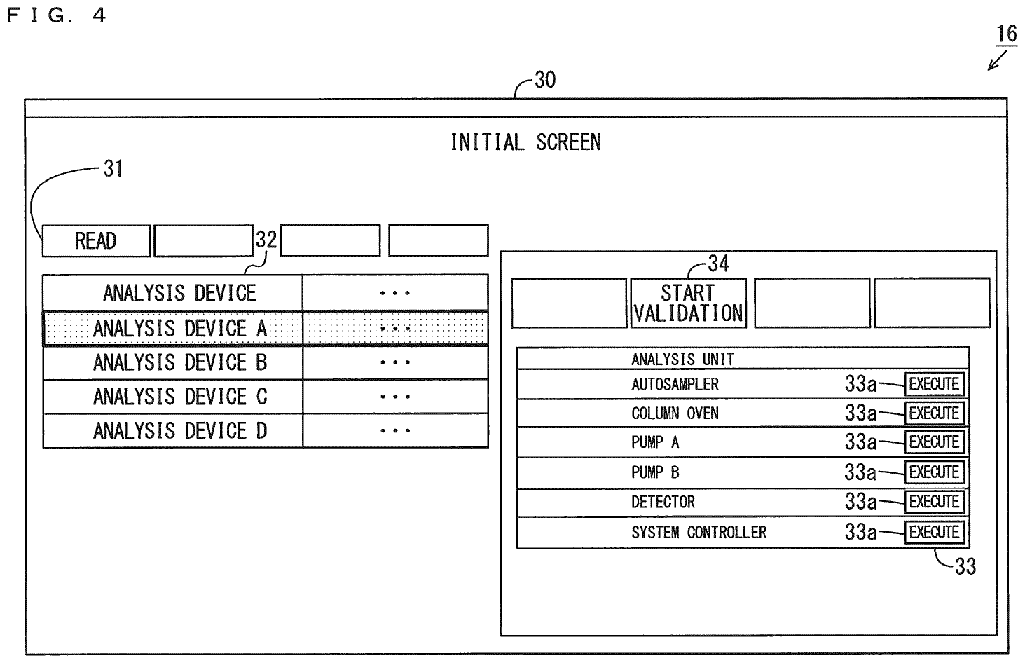

FIG. 4 is a diagram showing one example of an initial screen displayed in the display 16 in the validation process. As shown in FIG. 4, the initial screen 30 includes a read button 31, a device display field 32, a unit display field 33 and a start button 34. The performer can designate device information stored in the storage 14 of FIG. 1 by operating the read button 31 using the operation unit 15 of FIG. 1. In a case where the device information is designated, one or more analysis devices 20 represented by the device information are displayed in the device display field 32.

The performer can select a desired analysis device 20 among the analysis devices 20 displayed in the device display field 32 using the operation unit 15. In a case where an analysis device 20 is selected, one or more analysis units 21 included in the analysis device 20 are displayed in the unit display field 33. Further, one or more execution buttons 33a respectively corresponding to the one or more analysis units 21 are displayed in the unit display field 33.

The performer can operate the start button 34 using the operation unit 15. In this case, all of the analysis units displayed in the unit display field 33 are to be inspected, and an inspection of all of the analysis units 21 displayed in the unit display field 33 in regard to inspection items is started. Alternatively, the performer can operate an execution button 33a corresponding to a desired analysis unit 21 using the operation unit 15. In this case, an analysis unit 21 corresponding to an operated execution button 33a is to be inspected, and an inspection of the analysis unit 21 in regard to an inspection item is started.

FIG. 5 is a diagram showing one example of an execution screen 40 for an inspection in the validation process. The start button 34 of FIG. 4 is operated, whereby the initial screen 30 is put in an inactive state, and the execution screen 40 of FIG. 5 is displayed in the display 16. In the example of FIG. 5, the execution screen 40 includes a unit display field 41. In the unit display field 41, a plurality of analysis units 21, the inspection items for each analysis unit 21 being inspected, their progresses, etc. are displayed. In a case where the validation device 1 is waiting for a manual operation of an analysis unit 21 by the performer, such a notice is displayed in the unit display field 41.

Further, in the unit display field 41, one or more execution buttons 41a respectively corresponding to the one or more analysis units 21 are displayed. The performer operates an execution button 41a corresponding to a desired analysis unit 21 using the operation unit 15, thereby being able to individually instruct the validation device 1 to interrupt or retry an inspection of the analysis unit 21 in regard to an inspection item. Further, in the example of FIG. 5, one or more simulation screens 50 respectively simulating display panels b of one or more analysis units 21 are displayed in the display 16. In each simulation screen 50, the same screen as a screen displayed in a display panel b of a corresponding analysis unit 21 is displayed.

FIG. 6 is a diagram showing another example of an execution screen 40 for an inspection in the validation process. In the example of FIG. 6, two execution buttons 33a of FIG. 4 respectively corresponding to two pumps which are analysis units 21 are operated, so that the execution screen 40 is displayed in the display 16. The execution screen 40 of FIG. 6 is similar to the execution screen 40 of FIG. 5. Further, in the example of FIG. 6, a simulation screen 50 similar to the simulation screen 50 of FIG. 5 is displayed in the display 16.

As shown in the item of progress in the unit display field 41 of FIG. 5 or 6, inspections of a plurality of analysis units 21 can be executed simultaneously and in parallel. Here, priority levels indicating the order of inspections of the analysis units 21 may be included in device information. In this case, a specific analysis unit 21 can be inspected before or after other analysis units 21.

For example, a system controller is used to control the other analysis units 21 among the plurality of analysis units 21. Therefore, an inspection of the system controller is preferably executed first. Further, an autosampler among the plurality of analysis units 21 behaves after behavior conditions of the other analysis units 21 are determined. Therefore, an inspection of the autosampler is preferably executed last. Inspections of the plurality of analysis units 21 except for specific analysis units 21 such as the system controller and the autosampler may be executed simultaneously and in parallel.

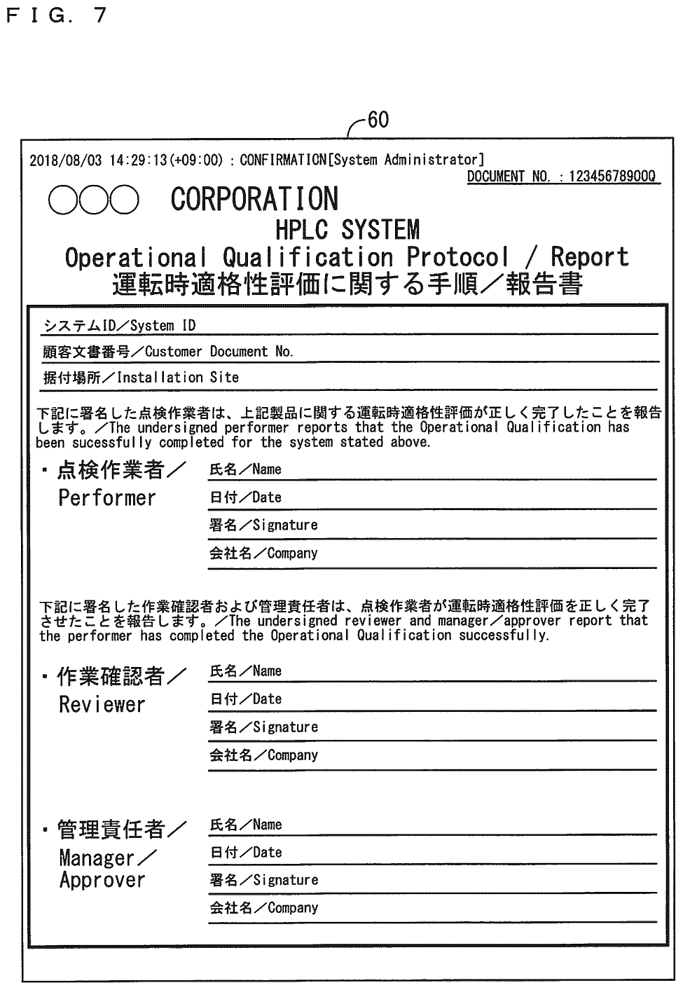

After the inspections of all of the analysis units 21 to be inspected end, a report showing the results of inspections is created automatically. FIG. 7 is a diagram showing one example of a report representing results of inspections. The report 60 is an electronic PDF file, for example, and only the first page of the report 60 is shown in FIG. 7. The report 60 is created by transfer of a result of inspection, the name of a performer, inspection execution date and time and so on to a predetermined format. Therefore, it is not necessary for the performer to create a report manually. This reduces time and labor for the performer. Further, an error in transferring a result of inspection and so on can be prevented.

### (3) Validation Process

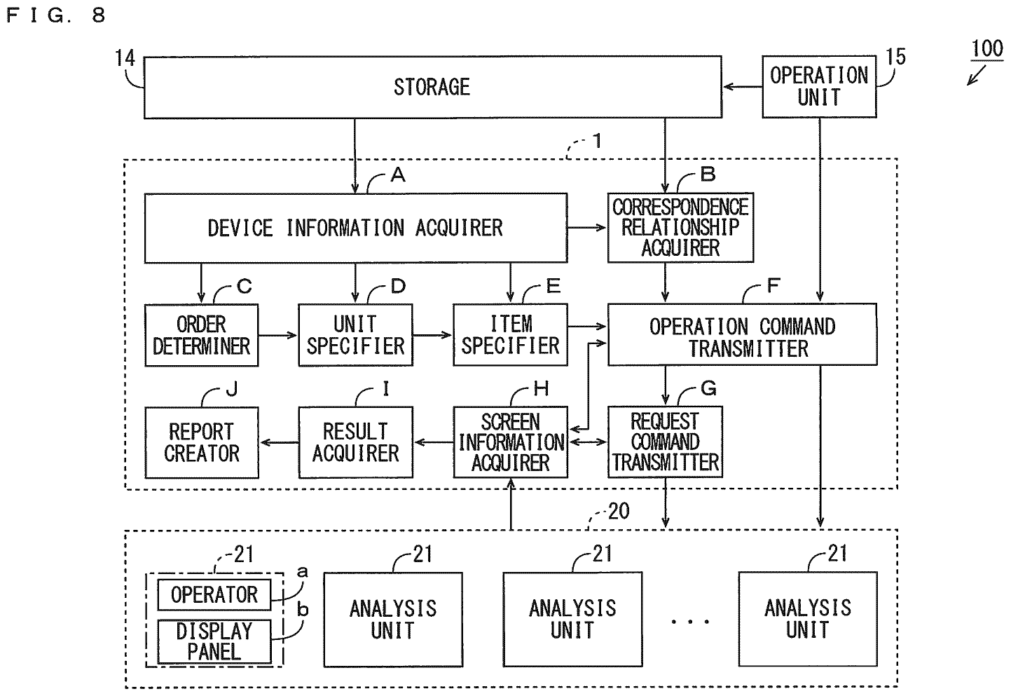

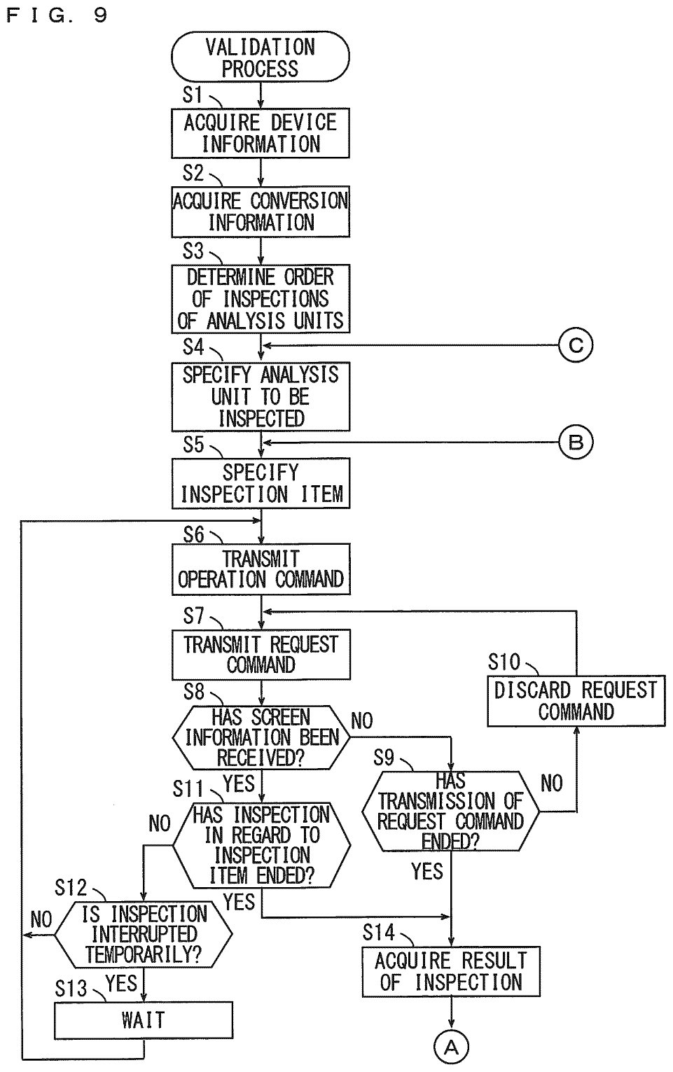

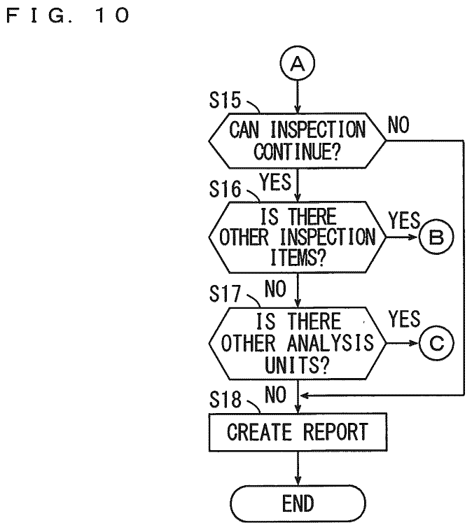

FIG. 8 is a diagram showing the configuration of the validation device 1. FIGS. 9 and 10 are flowcharts showing the algorithm of the validation process to be executed by the validation program. As shown in FIG. 8, the validation device 1 includes a device information acquirer A, a correspondence relationship acquirer B, an order determiner C, a unit specifier D, an item specifier E, an operation command transmitter F, a request command transmitter G, a screen information acquirer H, a result acquirer I and a report creator J as functions.

The CPU 11 of FIG. 1 executes the validation program stored in the storage 14 or the like, whereby the functions of the validation device 1 are implemented. Part or all of the functions of the validation device 1 may be implemented by hardware such as an electronic circuit. The validation process will be described below with reference to the validation device 1 of FIG. 8 and the flowcharts of FIGS. 9 and 10.

First, the device information acquirer A acquires designated device information among the device information stored in the storage 14 (step S1). In the initial screen 30 displayed in the display 16 of FIG. 4, the performer operates the read button 31 using the operation unit 15, and then can designate desired device information from among the device information stored in the storage 14 of FIG. 4 by performing a predetermined operation. The correspondence relationship acquirer B acquires conversion information corresponding to an analysis unit 21 included in the device information acquired in the step S1 (step S2).

The order determiner C determines the order of inspections of the analysis units 21 based on priority levels included in the device information acquired in the step S1. The unit specifier D specifies an analysis unit 21 to be inspected among the analysis units 21 included in the device information acquired in the step S1 based on the order determined in the step S3.

The item specifier E specifies an inspection item for the analysis unit 21 specified in the step S4 based on the device information acquired in the step S1 (step S5). The processes from the step S5 to the below-mentioned step S16 for a plurality of analysis units 21 having the same priority level are executed simultaneously and in parallel.

Next, the operation command transmitter F generates an operation command based on the correspondence relationship on the basis of the conversion information acquired in the step S2 and the inspection item specified in the step S5 and transmits the generated operation command to the analysis unit 21 (step S6). Subsequently, the request command transmitter G generates a request command for requesting screen information and transmits the generated request command to the analysis unit 21 (step S7). The screen information acquirer H determines whether the screen information responding to the request command transmitted in the step S7 has been received from the analysis unit 21 in a certain period of time (step S8).

In a case where the screen information is not received in the certain period of time by the screen information acquirer H, the request command transmitter G determines whether to end transmission of a request command (step S9). Here, in a case where the steps S7 to S9 and the below-mentioned step S10 are executed a predetermined number of times, the request command transmitter G determines to end transmission of a request command. In a case where ending transmission of a request command, the request command transmitter G interrupts an inspection and proceeds to the step S14.

In a case where not ending transmission of a request command, the request command transmitter G discards the request command transmitted in the step S7 (step S10) and returns to the step S7. In this case, in the step S7, the request command transmitter G retransmits a request command to the analysis unit 21.

In a case where the screen information is received in the certain period of time by the screen information acquirer H in the step S8, it is confirmed that the transmission command transmitted in the step S6 is received by the analysis unit 21 specified in the step S4. Here, in a case where a simulation screen 50 of FIG. 5 or FIG. 6 is displayed in the display 16, the screen information acquirer H causes the simulation screen 50 to display a screen based on the acquired screen information.

Next, the screen information acquirer H determines whether an inspection in regard to an inspection item has ended based on the screen information (step S11). In a case where the inspection in regard to the inspection item has not ended, the operation command transmitter F determines whether to interrupt the inspection temporarily (step S12). Here, in a case where the performer manually operates the analysis unit 21 during the inspection in regard to the inspection item, the operation command transmitter F determines to interrupt the inspection temporarily.

In a case where interrupting the inspection temporarily, the operation command transmitter F displays such a notice in the unit display field 41 of the execution screen 40 of FIGS. 5 and 6, waits until a manual operation of the analysis unit 21 by the performer ends (step S13) and returns to the step S6. The performer can notify the operation command transmitter F that the manual operation of the analysis unit 21 has ended, by operating an execution button 41a in the unit display field 41 in the operation unit 15. Also in a case where not interrupting the inspection temporarily in the step S12, the operation command transmitter F returns to the step S6. Thus, the inspection in regard to the inspection item continues.

In a case where the inspection in regard to the inspection item ends in the step S11, the screen information acquirer H proceeds to the step S14. In the step S14, the result acquirer I acquires a result of inspection in regard to the inspection item specified in the step S5 (step S14). Here, in a case where the process proceeds from the step S11 to the step S14, the result of inspection in regard to the inspection item indicating whether the analysis unit 21 has “passed” or “failed” the inspection is acquired based on the screen information received in the step S8. On the other hand, in a case where the process proceeds from the step S9 to the step S14, the result of inspection in regard to the inspection item indicating the analysis unit 21 has “failed” the inspection is acquired.

Then, the result acquirer I determines whether the inspection can continue based on the acquired result of inspection (step S15). Here, in a case where the result of inspection of an analysis unit 21 having a high priority level is “failed,” it is determined that the inspection cannot continue. In a case where the inspection cannot continue, the result acquirer I proceeds to the step S18. In a case where the inspection can continue, the result acquirer I determines whether there is any other inspection item (step S16).

In a case where there is another inspection item, the result acquirer I returns to the step S5. In this case, the other inspection item for the same analysis unit 21 is specified in the step S5. In a case where there is no other inspection item, the result acquirer I determines whether there is any other analysis unit 21 (step S17). In a case where there is another analysis unit 21, the result acquirer I returns to the step S4. In this case, the other analysis unit 21 to be inspected is specified in the step S4. In a case where there is no other analysis unit 21, the result acquirer I proceeds to the step S18.

In the step S18, the report creator J creates a report 60 based on a result of inspection acquired in the step S14 (step S18) and ends the validation process. Here, in a case where the process proceeds from the step S17 to the step S18, the created report 60 mentions results of inspections of all of the analysis units 21 in regard to the inspection items included in the device information acquired in the step S1. On the other hand, in a case where the process proceeds from the step S15 to the step S18, the created report 60 does not mention a result of inspection of part of the analysis units 21 in regard to an inspection item.

### (4) Effects

In the validation device 1 according to the present embodiment, an inspection item for an analysis unit is specified by the item specifier E. The correspondence relationship between an inspection item and an operator code assigned to each operator ‘a’ of an analysis unit 21 is acquired by the correspondence relationship acquirer B. Operation commands assigned to operators ‘a’ to be operated for an inspection of an analysis unit 21 are sequentially transmitted to an analysis unit 21 by the operation command transmitter F based on an inspection item and a correspondence relationship. A request command requesting transmission of screen information representing a screen to be displayed in the display panel b is transmitted to an analysis unit 21 by the request command transmitter G. The screen information transmitted by an analysis unit 21 in response to a request command is acquired by the screen information acquirer H.

Each time an operation command is transmitted to an analysis unit 21 by the operation command transmitter F, a next request command is transmitted to the analysis unit 21 by the request command transmitter G. Further, each time an operation command transmitted by the operation command transmitter F is received by an analysis unit 21, a screen displayed in the display panel b is updated based on the received operation command.

With this configuration, it is not necessary for the performer who manages an inspection of an analysis unit 21 to manually perform at least part of work for inspecting the analysis unit 21. This reduces time and labor for validating the analysis device 20. Further, the performer can identify that operation commands are sequentially received by an analysis unit 21 and the procedure for inspecting the analysis unit 21 proceeds in response to the received operation commands, by viewing the transition of screens displayed in the display panel b of the analysis unit 21. Therefore, whether the same inspection as the inspection to be executed manually has been executed properly can be easily confirmed. As a result, it is possible to maintain reliability of validation while reducing time and labor for validating the analysis device 20.

Further, a next operation command is transmitted to an analysis unit 21 while it is confirmed that an operator ‘a’ corresponding to each operation command has been operated. Thus, even in a case where a response from an analysis unit 21 is delayed, a series of operation commands can be transmitted to the analysis unit 21 easily and sequentially while an operator ‘a’ is being operated.

### (5) Another Embodiment

While the validation device 1 includes the result acquirer I and the report creator J in the above-mentioned embodiment, the present invention is not limited to this. In a case where the performer identifies a result of inspection and creates a report manually, a validation device 1 does not have to include a result acquirer I or a report creator J.

1. A method for analyzing a chromatograph using a validation device, the method comprising:
displaying on a display panel of the chromatograph selectable properties of the analysis components, wherein:
the chromatograph comprises a system controller, the display panel, and analysis components; and
the analysis components include a pump, an autosampler, a column oven, and a detector;
displaying on a display screen of the validation device selectable inspection items, wherein the validation device comprises an electronic device;
receiving by the central processor of the validation device a selected inspection item of the displayed selectable inspection items;
implementing an inspection of the selected inspection item by:
determining with the central processor an operator code associated with the selected inspection item stored in a memory of the validation device, wherein:
the memory stores operator codes in association with the selectable inspection items, such that each of the selectable inspection items is associated with an operator code of the operator codes; and
sending with the central processor the determined operator code to the chromatograph as an operation command, wherein the operation code identifies one of the displayed selectable properties of the analysis components;
transmitting to the chromatograph with the central processor a request command to the chromatograph requesting transmission to the validation device of the displayed result of the performed inspection;
receiving with the system controller of the chromatograph the sent operation command;
performing with the system controller an inspection of the one of the displayed selectable properties of the analysis components identified by the received operation command; and
displaying on the display panel of the chromatograph a result of the performed inspection;
receiving by the system controller the sent request command;
sending with the system controller the displayed result to the validation device;
receiving by the central processor the requested displayed result; and
displaying on the display screen the received requested displayed result.

2. The method according to claim 1, wherein, after transmitting the request command by the central processor and until the requested displayed result is received by the central processor, the method additionally includes repeatedly:
determining with the central processor when a time threshold has passed, wherein the time threshold has passed when an amount of time since the request command was transmitted to the chromatograph exceeds the time threshold and the requested displayed result has not been received; and
when the time threshold has passed, retransmitting to the chromatograph by the central processor the request command to the chromatograph.

3. The method according to claim 1, wherein:
the chromatograph is one of a plurality of chromatographs;
each of the selectable inspection items displayed on the display screen is associated with one of the plurality of chromatographs;
the central processor receives multiple selected inspection items from the displayed selectable inspection items, wherein the received multiple inspection items are together associated with more than one of the plurality of chromatographs;
the method further includes:
receiving priority levels for the plurality of chromatographs indicating the order of inspections of the plurality of chromatographs;
determining an order of inspection of the more than one of the plurality of chromatographs based on the received priority levels; and
implementing the inspection of the more than one of the plurality of chromatographs based on the determined order of inspections.

4. The method according to claim 1, further comprising generating and outputting by the central processor a report based on the received requested display result.

5. An analysis system comprising:
a chromatograph comprising a system controller, a display panel, and analysis components including a pump, an autosampler, a column oven, and a detector, wherein the system controller is configured to:
display on the display panel selectable properties of the analysis components;
receive a selected property of the displayed selectable properties of the analysis components;
perform an inspection of the received selected property; and
display on the display panel a result of the performed inspection;
a validation device comprising an electronic device for inspecting the chromatograph including:
a display screen configured to display selectable inspection items;
a memory storing operator codes in association with the selectable inspection items, such that each of the selectable inspection items is associated with an operator code of the operator codes;
a central processor configured to execute an inspection of the chromatograph by:
displaying on the display screen the selectable inspection items;
receiving a selected inspection item of the displayed selectable inspection items;
determining an operator code associated with the selected inspection item stored in the memory; and
sending the determined operator code to the chromatograph as an operation command, wherein the operation code identifies one of the displayed selectable properties of the analysis components;
wherein the system controller of the chromatograph is further configured to:
receive the sent operation command;
perform the inspection of the one of the displayed selectable properties of the analysis components identified by the received operation command; and
display on the display panel the result of the performed inspection;
wherein the execution of the inspection of the chromatograph by the central processor further includes:
after sending the determined operator code, transmitting to the chromatograph a request command to the chromatograph requesting transmission to the validation device of the displayed result of the performed inspection;
receiving the requested displayed result; and
displaying on the display screen the received result requested displayed result; and
wherein the system controller is further configured to:
receive the sent request command; and
send the displayed result to the validation device.

6. The analysis system according to claim 5, wherein, after transmitting the request command and until the requested displayed result is received, the execution of the inspection of the chromatograph by the central processor additionally includes repeatedly:
determining when a time threshold has passed, wherein the time threshold has passed when an amount of time since the request command was transmitted to the chromatograph exceeds the time threshold and the requested displayed result has not been received; and
when the time threshold has passed, retransmitting to the chromatograph the request command to the chromatograph.

7. The analysis system according to claim 5, further comprising a plurality of chromatographs, wherein:
the chromatograph is one of the plurality of chromatographs;
the execution of the inspection of the chromatograph by the central processor further includes:
displaying the selectable inspection items, such that each of the selectable inspection items is associated with one of the plurality of chromatographs;
receive multiple selected inspection items from the displayed selectable inspection items, wherein the received multiple inspection items are together associated with more than one of the plurality of chromatographs;
receiving priority levels for the plurality of chromatographs indicating the order of inspections of the plurality of chromatographs; and
determining an order of inspection of the more than one of the plurality of chromatographs based on the received priority levels; and
executing the inspection of the more than one of the plurality of chromatographs based on the determined order of inspections.

8. The analysis system according to claim 5, wherein the central processor is further configured to generate and output a report based on the received requested display result.
